(19) United States
(12) Patent Application Publication
(10) Pub. No.: US 2023/0377219 A1
(43) Pub. Date: Nov. 23, 2023

(54) SYSTEMS AND METHODS FOR RECONSTRUCTION AND VISUALIZATION OF ANATOMICAL DATA

(71) Applicant: OneProjects Design and Innovation Ltd., Dublin (IE)

(72) Inventors: Christoph Hennersperger, München (DE); Jakob Weiss, Munchen (DE)

(21) Appl. No.: 18/198,548

(22) Filed: May 17, 2023

Related U.S. Application Data
(60) Provisional application No. 63/343,771, filed on May 19, 2022.

(51) Int. Cl. G06T 11/00; A61B 8/12; A61B 8/08; A61B 8/00

(52) U.S. Cl. CPC G06T 11/003; A61B 8/12; A61B 8/483; A61B 8/4488

(57) ABSTRACT

The invention provides systems and methods providing reconstruction of three-dimensional (3D) ultrasound image data into a combination of multiple views of an anatomical region of interest, wherein the multiple views including both 3D views and two-dimensional (2D) views spatially related to one another.

### CROSS-REFERENCE TO RELATED APPLICATIONS

This application claims priority to, and the benefit of, U.S. Provisional Application No. 63/343,771, filed on May 19, 2022, the content of which is incorporated by reference herein in its entirety.

### TECHNICAL FIELD

The disclosure relates to visualization of biological tissue, and, more particularly, to systems and methods providing reconstruction of three-dimensional (3D) ultrasound image data into a combination of multiple views of an anatomical region of interest associated with intravascular and/or intracardiac tissue, wherein the multiple views include both 3D views and two-dimensional (2D) views spatially related to one another.

### BACKGROUND

Medical imaging refers to several different technologies that are used to view the human body in order to diagnose, monitor, or treat medical conditions. As such, medical imaging is generally recognized as one of the most powerful diagnostic and intervention tools in medicine. The most common types of medical imaging modalities include, but are not limited to, x-ray imaging, Magnetic Resonance Imaging (MM), and ultrasound (US) imaging. While each type of imaging modality has its particular advantages, as well as its associated drawbacks, ultrasound is becoming a more common imaging technique, due in large part to its portability, ease of use, noninvasiveness, and reduced costs, when compared to other imaging modalities.

Ultrasound imaging is a medical imaging technique for imaging organs and soft tissues in a human body. Ultrasound imaging uses non-invasive high frequency sound waves to produce an ultrasound image. The ultrasound image is produced based on the reflection of the waves off of the body structures. The strength (amplitude) of the sound signal and the time it takes for the wave to travel through the body provide the information necessary to produce an image.

Ultrasound imaging can help a physician evaluate, diagnose and treat various medical conditions. When making a diagnosis based on an ultrasound examination, physicians must rely on adequate image quality, acquisition of proper views, and sufficient quantification of all relevant structures and flows.

For example, ultrasound imaging may generally be in the form of two-dimensional (2D) or three-dimensional (3D) imaging. Conventional 2D ultrasound imaging has been widely used because it can dynamically display 2D images of the region of interest in real-time. However, due to the lack of the anatomy and orientation information, clinicians have to imagine the volume with the planar 2D images mentally when they need the view of 3D anatomic structures. In order to address the drawbacks of 2D ultrasound imaging, 3D ultrasound imaging was developed to help the diagnosticians acquire a full understanding of the spatial anatomic relationship. In particular, physicians can view an arbitrary plane of the reconstructed 3D volume, as well as panoramic view of the region of interest, which is intended to help surgeons ascertain whether a surgical instrument is placed correctly within the region of interest.

While advancements in ultrasound imaging technology have provided some improvements, current ultrasound imaging systems still have drawbacks. In particular, it has been found that the various views provided by 3D ultrasound imaging systems pose an unfamiliar representation for the clinician. As such, 2D ultrasound imaging still remains the natural go-to imaging modality for many clinicians. Yet, as previously described, a major drawback of 2D ultrasound imaging is the limited field of view provided, as well as the required manual interaction with the probe or catheter, which is error prone, cumbersome, and generally challenging for users.

### SUMMARY

The present invention recognizes the drawbacks of current ultrasound imaging systems, namely the limited field of view and required interaction associated with 2D imaging systems and the complexities of navigating and working with 3D imaging systems. The present invention provides an imaging system configured to provide the benefits of both 2D and 3D imaging technologies without the associated drawbacks, thereby providing a user (clinician or the like) with optimal orientation and navigation capabilities when performing ultrasound-related procedures.

Aspects of the invention may be accomplished using an imaging system configured to reconstruct 3D ultrasound image data, specifically 3D volumetric data, into a combination of multiple views of an anatomical region of interest, wherein the multiple views include both 3D views and 2D views spatially related to one another. The imaging system is configured to provide a user with seamless interaction with both 2D and 3D imaging views, in which a user can select one or more 2D images to be dynamically reconstructed from 3D volumetric data. The multiple 2D views are directly related to the 3D view, as the 2D views are digitally reconstructed from the volume data itself. The imaging system provides the user with an interactive interface, in which the user is able to specifically select a specific portion of a region of interest depicted in a 3D view from which related 2D images are reconstructed. As such, the multiple views (2D and 3D views) are related to the same anatomical region of interest. The imaging system is further configured to annotate at least the 3D view so as to highlight those specific portions of the region of interest that have been reconstructed into 2D views.

Furthermore, in some embodiments, the system is capable of providing a user with the option of seamlessly switching between various 2D views. In particular, a user may be able to utilize the interactive interface to switch to a 2D view of interest without requiring reconstruction from a 3D view in which the 2D view of interest may have the same or a different imaging configuration. For example, a given workflow may involve a user selecting a digitally reconstructed (and/or digitally steered via a manipulatable probing device) view from 3D volumetric data, and the user may subsequently switch, via the interactive interface, to a selected 2D view of interest providing a different viewing mode (e.g., a flow imaging mode or the like).

Accordingly, the system provides a user with an intuitive understanding of the interrelation of views and orientation within the acquired 3D data, thereby overcoming the limitations plaguing current imaging systems (i.e., 2D and 3D US imaging), while providing the advantages associated with each. In particular, the relationship between 2D and 3D views mimics current clinical imaging probes (e.g. linear/curvilinear/phased/rotational) in both appearance as well as interaction. In particular, users can digitally steer and/or navigate the 2D views (i.e., by way of interacting with the 3D volume via an interface) without requiring the manual maneuvering of the ultrasound probe, which can be cumbersome and prone to error.

One aspect of the present invention includes an ultrasound imaging system that includes a console configured to be operably associated with an imaging device. The console comprises a hardware processor coupled to non-transitory, computer-readable memory containing instructions executable by the processor to cause the console to: receive three-dimensional (3D) image data from an ultrasound imaging device; and dynamically reconstruct multiple images from the 3D image data, the multiple images comprising at least one 3D image providing a 3D view of an anatomical region of interest and one or more corresponding two-dimensional (2D) images providing 2D views of the anatomical region of interest and spatially related to the 3D view.

Each of the multiple images may include at least one of a slice-based image and a volume-based image. For example, in one embodiment, the at least one 3D image provides a full circumferential 360-degree visualization of the anatomical region of interest. In such an embodiment, the systems and methods described herein are included with a catheter-based system (or system utilizing a probe of some sort). The at least one 3D image may provide an orbital volume visualization of the anatomical region of interest. In some embodiments, the one or more 2D images provides at least one of a phased array visualization and circumferential radial visualization of the anatomical region of interest. Accordingly, in some embodiments, a user may be presented with a 3D (providing an orbital volume visualization of the anatomical region of interest) and two or more 2D images (a first 2D image providing a phased array visualization of the anatomical region of interest and a second 2D image providing a circumferential radial visualization of the anatomical region of interest).

It should be noted that, in some embodiments, some of the 2D images may be reconstructed from data acquired by a co-located, secondary imaging modality, in addition, or alternatively, to being reconstructed from the 3D mage data. For example, in some embodiments, systems and methods of the present invention may further make use of a secondary imaging modality, such as a computed tomography (CT) imaging system, a transmission imaging system, a brightfield or darkfield imaging system, a fluorescence imaging system, a phase contrast imaging system, a differential interference contrast imaging system, a hyperspectral imaging system, a Raman or surface-enhanced Raman imaging system, and a magnetic resonance imaging (MRI) system. For example, in some embodiments, one or more 2D images may be reconstructed based pre-registered CT volume data, MRI volume data, etc.

In some embodiments, each of the multiple images comprises one or more annotations providing a visual indication of a spatial relationship of one of the multiple images relative to another one of the multiple images. The one or more annotations may include a highlighted marking or the like.

For example, in some embodiments, the multiple images comprise a 3D image and two 2D images spatially related to the 3D image. In such an embodiment, the 3D image comprises two annotations separately associated with each of the two 2D images, wherein each of the two annotation provides a visual indication of a position of the anatomical region of interest from within the 3D view that is associated with a 2D view of a respective 2D image. Yet still, in some embodiments, each of the 2D images comprises an annotation associated with one another. For example, an annotation in a first one of the 2D images (e.g., 2D image providing a phased array visualization) provides a visual indication of a position of the anatomical region of interest from within the 2D view of the first one of the 2D images that is associated with the 2D view of a second one of the 2D images (e.g., 2D image providing a circumferential radial visualization). Accordingly, an annotation in the second one of the 2D images provides a visual indication of a position of the anatomical region of interest from within the 2D view of the second one of the 2D images that is associated with the 2D view of the first one of the 2D images.

In some embodiments, the console may be configured to augment the multiple images with the one or more annotations based, at least in part, on user input with the system. For example, the user may be able to select the specific portion of the region of interest from which the 2D images are to be reconstructed and further select whether the spatial relationship between the 2D and 3D images is to be highlighted. In some embodiments, the system may be configured to provide suggested views to the user, such as suggested 2D views that correspond to anatomical landmarks of interest. For example, in a catheter-based application, a catheter may be positioned within a default, home view within an anatomical region of interest (e.g., the right atrium) wherein relevant structures in this home view (e.g. tricuspid valve, mitral annulus, fossa ovalis, etc.) may be automatically recognized and suggested, via the system, as 2D views to be displayed. The suggested 2D views could also correspond to typical, standard views commonly presented in current 2D imaging systems to thereby provide an even more intuitive interaction and experience for the user.

An interactive interface operably associated with the console may further allow a user to select the type of annotation (i.e., select color, visibility, intensity, blurring, or the like) for highlighting the relationship between images. By providing a user with the ability to freely select a visual cue of their choice, the system of the present invention enables a more intuitive relationship and interpretation by the user. In particular, the system allows for a more customizable approach to providing visualization of anatomical regions of interest.

In some embodiments, the console is configured to receive the full circumferential, 3D image data in real-time or near real-time and the console is configured to reconstruct the multiple images in real-time or near real-time, based, at least in part, on user input and/or predefined protocols.

In some embodiments, the ultrasound imaging device comprises a catheter-based ultrasound imaging device comprising a catheter including a rotatable ultrasound transducer array provided thereon configured to transmit ultrasound pulses to, and receive echoes of the ultrasound pulses from, intravascular and/or intracardiac tissue. Accordingly, the image data may be in the form of reflected signal data based on received echoes of the ultrasound pulses from the intravascular and/or intracardiac tissue. Yet still, in some embodiments, the ultrasound imaging device comprises a transesophageal echocardiogram (TEE) probe, for example. As such, 3D image data collected via a catheter-based or probe-based ultrasound imaging device may provide a full circumferential 360-degree visualization of the anatomical region of interest.

It should be noted that, in some embodiments, non-circumferential image data may be collected. For example, the systems and methods of the present invention may utilize a four dimensional (4D) system (with a pyramidal field of view).

In some embodiments, the console is further configured to: process the reflected signal data using at least one of a functional imaging algorithm and an anatomical imaging algorithm to extract associated functional and anatomical parameter data of the anatomical region of interest and reconstruct the multiple images from the extracted functional and/or anatomical parameter data; and output, via a display, the reconstructed multiple images to an operator depicting visualization of the anatomical region of interest. The functional parameter data may include, but is not limited to, at least one of tissue perfusion, tissue motion, physiological motion, tissue stiffness or elasticity, tissue strain, tissue anisotropy, tissue coherence, specific statistic tissue parameters modeled by statistical distributions, textural parameters of the tissue, and spectral and frequency-based parameters of the tissue, as well as fluid flow (i.e., blood flow and the like).

The functional parameter data may be indicative of a characterization of tissue at the anatomical region of interest and the anatomical parameter data comprises at least one of spatial and geometrical relationship of tissue at the anatomical region of interest. The tissue characterization may include at least one of tissue type, tissue health, tissue depth, lesion formation in the tissue as a result of an ablation procedure, and lesion depth in the tissue.

### BRIEF DESCRIPTION OF THE DRAWINGS

FIG. 1 is a diagrammatic illustration of a medical imaging system for providing reconstruction of 3D ultrasound image data into a combination of multiple views (2D and 3D images) of an anatomical region of interest associated with intravascular and/or intracardiac tissue.

FIG. 2 is a block diagram illustrating exchange of data between the imaging device, console unit, and display of the medical imaging system consistent with the present disclosure.

FIG. 3 is a block diagram illustrating one embodiment of a method for reconstruction of 3D ultrasound image data into a combination of multiple views (2D and 3D images) of an anatomical region of interest consistent with the present disclosure.

FIG. 4 shows an exemplary display of multiple views of an anatomical region of interest in accordance with the systems and methods of the present disclosure, illustrating the presentation of a combination a 3D image and two 2D images spatially related to the 3D image.

FIG. 5 shows as the display of the multiple views of the anatomical region of interest, illustrating the 2D and 3D images augmented with annotations that provide visual indications of positions of the anatomical region of interest from which the 2D images are reconstructed.

### DETAILED DESCRIPTION

By way of overview, the present invention is directed to systems and methods providing reconstruction of three-dimensional (3D) ultrasound image data into a combination of multiple views of an anatomical region of interest associated with intravascular and/or intracardiac tissue, wherein the multiple views include both 3D views and two-dimensional (2D) views spatially related to one another.

In particular, the system of the present invention provides users (i.e., clinicians or the like) with an improved means with which to interact with and utilize ultrasound imaging technologies to evaluate, diagnose and treat various medical conditions. Aspects of the invention may be accomplished using an imaging system configured to reconstruct 3D ultrasound image data, specifically 3D volumetric data, into a combination of multiple views of an anatomical region of interest, wherein the multiple views include both 3D views and 2D views spatially related to one another. The imaging system is configured to provide a user with seamless interaction with both 2D and 3D imaging views, in which a user can select one or more 2D images to be dynamically reconstructed from 3D volumetric data. The multiple 2D views are directly related to the 3D view, as the 2D views are digitally reconstructed from the volume data itself. The imaging system provides the user with an interactive interface, in which the user is able to specifically select a specific portion of a region of interest depicted in a 3D view from which related 2D images are reconstructed. As such, the multiple views (2D and 3D views) are related to the same anatomical region of interest. The imaging system is further configured to annotate at least the 3D view so as to highlight those specific portions of the region of interest that have been reconstructed into 2D views.

Accordingly, the system of the present invention provides a user with an intuitive understanding of the interrelation of multiple views (i.e. both 2D and 3D views) and orientation within the acquired 3D data, thereby overcoming the limitations plaguing current imaging systems (i.e., 2D and 3D US imaging systems), while providing the advantages associated with each. In particular, the relationship between 2D and 3D views mimics current clinical imaging probes (e.g. linear/curvilinear/phased/rotational) in both appearance as well as interaction. In particular, users can digitally steer and/or navigate the 2D views (i.e., by way of interacting with the 3D volume via an interface) without requiring the manual maneuvering of the ultrasound probe, which can be cumbersome and prone to error.

It should be noted that the following description focuses on use of the present invention for ultrasound visualization of intravascular and/or intracardiac tissue, which may be particularly useful for catheter-based interventional procedures for assessing the anatomy as well as functional data in relation to the target volume of interest. However, as generally understood, the systems and methods of the present invention can be used for ultrasound visualization of tissue of any kind with respect to any kind of procedure in which imaging analysis is used and/or preferred. For example, in one embodiment, the systems and methods of the present invention can be particularly useful for catheter ablation procedures and classifying lesion formations associated therewith.

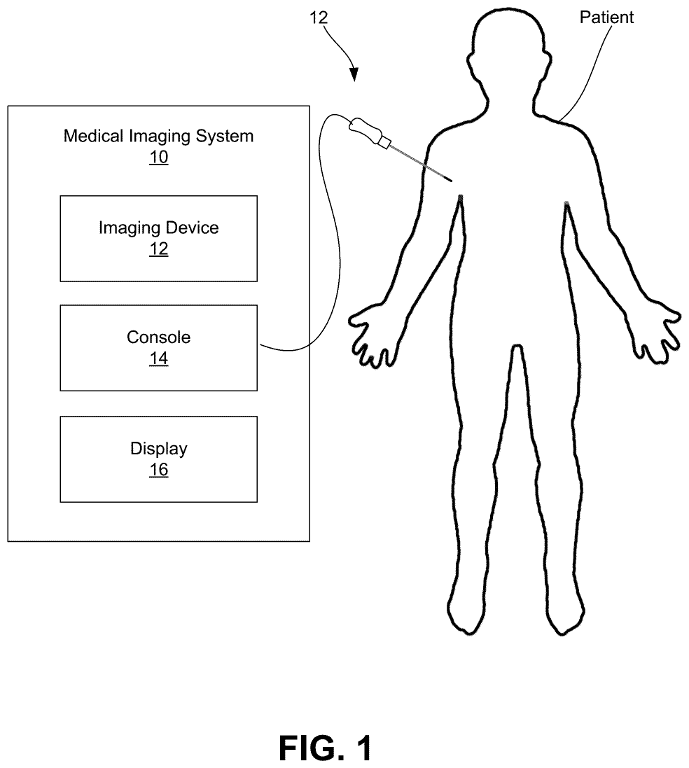

FIG. 1 is a diagrammatic illustration of an exemplary medical imaging system 10. In the illustrated embodiment, the medical imaging system 10 is an ultrasound system and includes an imaging device 12 operably coupled to a console 14 and a display 16. As generally understood, ultrasound imaging (sonography) uses high-frequency sound waves to view inside the body. Because ultrasound images are captured in real-time, they can also show movement of the body's internal organs as well as fluid flow (e.g., blood flowing through blood vessels). In an ultrasound exam, the ultrasound device 12, also referred to as a transducer probe, is placed directly on the skin or inside a body opening.

The ultrasound transducer probe 12 is responsible for sending and receiving the sound waves that create an ultrasound image via the piezoelectric effect, a phenomenon that causes quartz crystals within the probe to vibrate rapidly and send out sound waves. These waves then bounce off objects and are reflected to the probe.

It should be noted that, in the present invention, the probe 12 may use any type of transducer for transmitting and receiving acoustic waves. For example, the probe 12 may include one- or two-dimensional arrays of electronic transducer elements to transmit and receive acoustic waves. These arrays may include micro-electro-mechanical systems (MEMS)-based transducers, such as capacitive micro-machined ultrasound transducers (CMUTs) and/or piezoelectric micro-machined ultrasound transducers (PMUTs).

As generally understood, CMUT devices can offer excellent bandwidth and acoustic impedance characteristics, which makes them the preferable over conventional piezoelectric transducers. The vibration of a CMUT membrane can be triggered by applying pressure (for example using ultrasound) or can be induced electrically. The electrical connection to the CMUT device, often by means of an integrated circuit (IC) such as an application specific integrated circuit (ASIC), facilitates both transmission and reception modes of the device. In a reception mode, changes in the membrane position cause changes in electrical capacitance, which can be registered electronically while in a transmission mode, applying an electrical signal causes vibration of the membrane.

Regarding PMUT devices, unlike bulk piezoelectric transducers which use the thickness-mode motion of a plate of piezoelectric ceramic such as PZT or single-crystal PMN-PT, PMUT devices are based on the flexural motion of a thin membrane coupled with a thin piezoelectric film, such as PVDF. In comparison with bulk piezoelectric ultrasound transducers, PMUT devices can offer advantages such as increased bandwidth, flexible geometries, natural acoustic impedance match with water, reduced voltage requirements, mixing of different resonant frequencies and potential for integration with supporting electronic circuits especially for miniaturized high frequency applications.

The transducer probe 12 is operably coupled to a console 14, which is generally controls operation of the transducer probe 12 (i.e., transmission of sound waves from the probe). The console 14 may generally include one or more processors (e.g., a central processing unit (CPU), a graphics processing unit (GPU), or both) and storage, such as main memory, static memory, or a combination of both, which communicate with each other via a bus or the like. The memory according to the invention can include a machine-readable medium on which is stored one or more sets of instructions (e.g., software) embodying any one or more of the methodologies or functions described herein. The software may also reside, completely or at least partially, within the main memory and/or within the processor during execution thereof by the computer system, the main memory and the processor also constituting machine-readable media. The software may further be transmitted or received over a network via the network interface device.

For example, in an exemplary embodiment, the console 14 may generally include a computing device configured to communicate across a network. The computing device includes one or more processors and memory, as well as an input/output mechanism (i.e., a keyboard, knobs, scroll wheels, or the like) with which an operator can interact so as to operate the machine, including making adjustments to the transmission characteristics of the probe, saving images, and performing other tasks described herein, including selection of specific regions of interest for subsequent reconstruction into 2D and/or 3D images.

During operation, the CPU and/or GPU may control the transmission and receipt of electrical currents, subsequently resulting the emission and receipt of sound waves from the probe 12. The CPU and/or GPU also analyzes electrical pulses that the probe makes in response to reflected waves coming back and then converts this data into images (i.e., ultrasound images) that can then be viewed on a display 16, which may be an integrated monitor. Such images may also be stored in memory and/or printed via a printer (not shown).

In the illustrated embodiment, the imaging device 12 may generally be in the form of an imaging catheter capable of providing imaging and mapping capabilities. Accordingly, such a device 12 may be useful for ultrasound visualization of intravascular and/or intracardiac tissue, which may be particularly useful for catheter-based interventional procedures for assessing the anatomy as well as functional data in relation to a target volume of interest. As generally understood, the systems and methods of the present invention can be used for ultrasound visualization of tissue of any kind with respect to any kind of procedure in which imaging analysis is used and/or preferred.

By way of example, in one embodiment, the imaging device 12 may be useful in carrying out catheter ablation to treat a cardiac condition, such as atrial fibrillation (AF) or the like. For example, in some embodiments, the catheter 12 may further include additional components providing associated capabilities. For example, portions of the catheter may include sensors (e.g., localization and/or tracking sensors) and/or energy delivery elements (e.g., ablation elements). Accordingly, systems and methods of the present invention can be particularly useful for catheter ablation procedures and classifying lesion formations associated therewith. However, it should be noted that the systems and methods of the present invention can be useful for monitoring, diagnosing, and/or treating various conditions associated with a targeted region of interest and is not limited to intravascular and/or intracardiac tissue and conditions associated therewith.

The imaging catheter 12 may include a fully rotatable transducer unit comprised of an ultrasound transducer array configured to transmit ultrasound pulses to, and receive echoes of the ultrasound pulses from, surrounding intravascular tissue during a procedure. Such ultrasound transmissions result in a collection of image data which is received by the console 14 and subsequently reconstructed into one or more images providing visualization and characterization of the surrounding intravascular tissue. In particular, the console 14 may utilize image data received from an imaging assembly of the imaging catheter 12 to reconstruct one or more images, including at least 2D and 3D images of the anatomical region of interest (i.e., intravascular and/or intracardiac tissue). The console 14 may process the received image data utilizing certain imaging protocols and algorithms for reconstructing images and subsequently outputting, via a display, the reconstructed images to an operator depicting visualization of the anatomical region of interest. In addition to providing reconstruction of images based on received image data from the imaging assembly, the console 14 may further provide control over the imaging assembly, including control over the emission of ultrasound pulses therefrom (intensity, frequency, duration, etc.) as well as control over the movement of the ultrasound transducer unit (i.e., controlling rotation, including speed and duration of rotation).

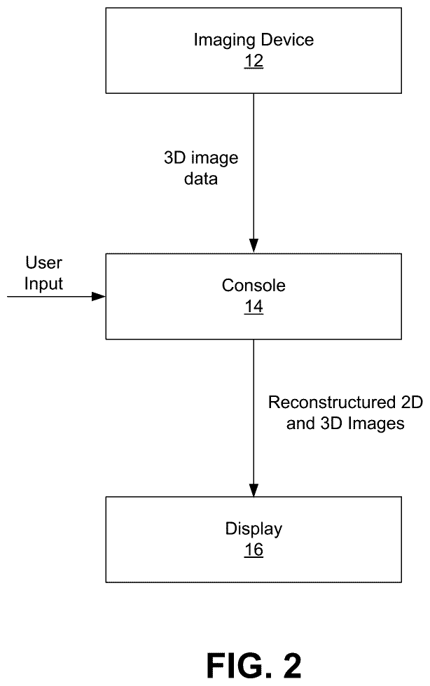

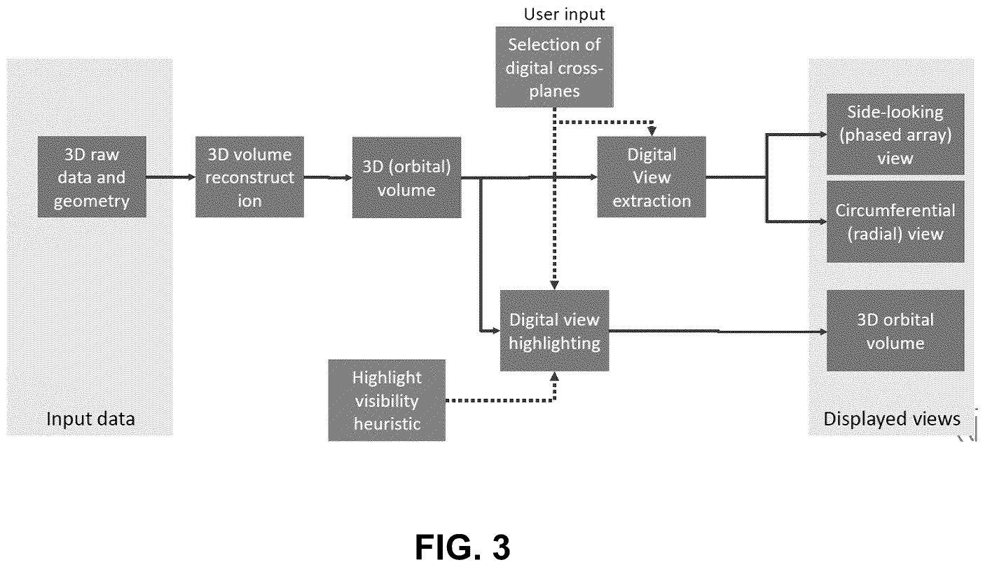

FIG. 2 is a block diagram illustrating exchange of data between the imaging device 12, console unit 14, and display 16 of the medical imaging system 10. FIG. 3 is a block diagram illustrating one embodiment of a method for reconstruction of 3D ultrasound image data into a combination of multiple views (2D and 3D images) of an anatomical region of interest.

As shown, the console unit 14 is configured to receive 3D image data, from the imaging device 12 and process such data to dynamically reconstruct multiple images. The 3D image data may generally include full circumferential, 3D image data, specifically 3D volumetric data, captured by the imaging device 12 during an ultrasound procedure. The console 14 is configured to reconstruct such data into at least one 3D image providing a 3D view of an anatomical region of interest and one or more corresponding 2D images providing 2D views of the anatomical region of interest and spatially related to the 3D view. The console 14 may be equipped with certain hardware and software for providing such image reconstruction and imaging assembly control, as described in International PCT Application No. PCT/IB2019/000963 (Published as WO 2020/044117) to Hennersperger et al., the content of which is incorporated by reference herein in its entirety.

In turn, the console 14 is configured to provide the multiple images (2D and 3D images) to the display 16, which presents the 2D and 3D views to a user, thereby depicting visualization of the anatomical region of interest.

The imaging system 10 provides the user with an interactive interface, in which the user is able to specifically select a specific portion of a region of interest depicted in a 3D view from which related 2D images are reconstructed. As such, the multiple views (2D and 3D views) are related to the same anatomical region of interest.

Accordingly, the imaging system 10 is configured to provide a user with seamless interaction with both 2D and 3D imaging views, in which a user can select one or more 2D images to be dynamically reconstructed from 3D volumetric data. The multiple 2D views are directly related to the 3D view, as the 2D views are digitally reconstructed from the volume data itself.

Each of the multiple images may include at least one of a slice-based image and a volume-based image. For example, in one embodiment, the at least one 3D image provides a 360-degree visualization of the anatomical region of interest. The at least one 3D image may provide an orbital volume visualization of the anatomical region of interest. In some embodiments, the one or more 2D images provides at least one of a phased array visualization and circumferential radial visualization of the anatomical region of interest. Accordingly, in some embodiments, a user may be presented with a 3D (providing an orbital volume visualization of the anatomical region of interest) and two or more 2D images (at least a first 2D image providing a phased array visualization of the anatomical region of interest and at least a second 2D image providing a circumferential radial visualization of the anatomical region of interest). It should be noted that, in some embodiments, a plurality of 2D views can be generated and displayed (e.g., 4- or 8-phased array slices reconstructed at certain angles).

It should be noted that in some embodiments, the system can be configured to provide continuous 3D image data. Additionally, or alternatively, the system can be configured to provide high-resolution 2D imaging data spatially aligned with one direction of a 360-degree view. For example, the system may generally operate in an alternative configuration, allowing for high-resolution (both temporal and spatial) 2D imaging in a single direction.

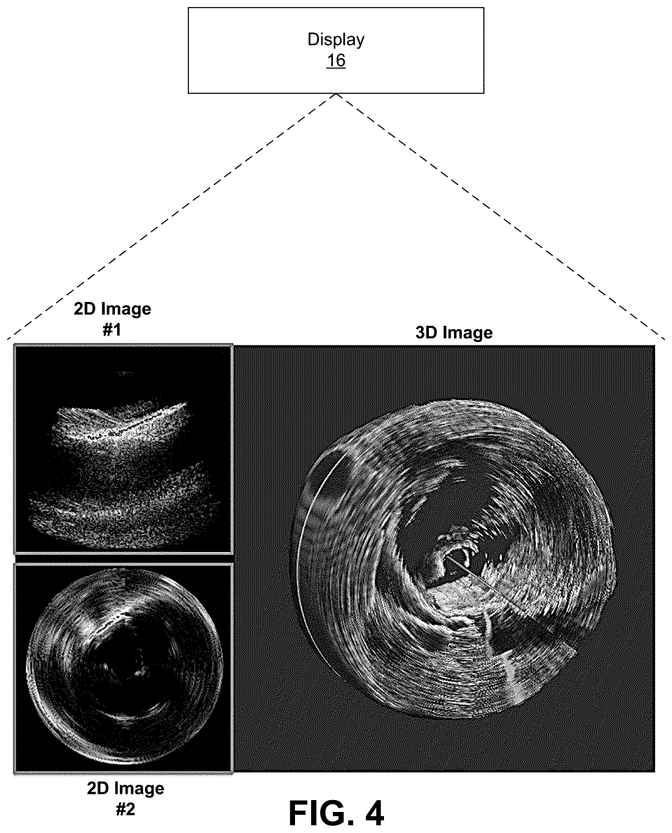

FIG. 4 shows one exemplary embodiment of a display of multiple views of an anatomical region of interest in accordance with the systems and methods of the present disclosure, illustrating the presentation of a combination a 3D image and two 2D images spatially related to the 3D image. As shown, the display 16 provides a user with a multiple view presentation of a phantom 3D volume visualization and corresponding plane view sections. The 2D plane views reconstructed with this approach are shown as phased array view (top left) and circumferential 2D view (bottom left).

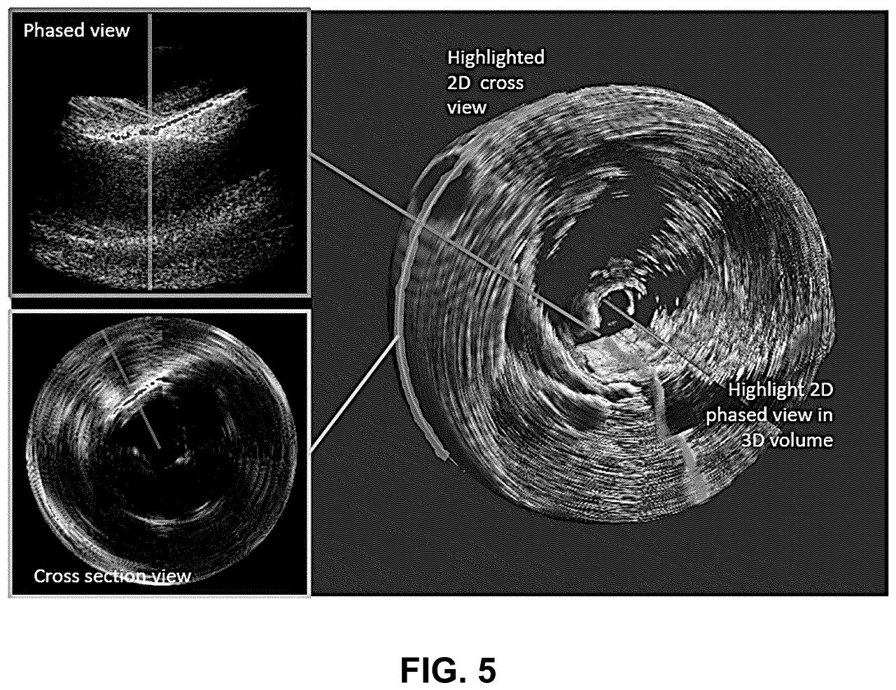

The imaging system 10 is further configured to annotate at least the 3D view so as to highlight those specific portions of the region of interest that have been reconstructed into 2D views. For example, FIG. 5 shows one exemplary embodiment of a display of the multiple views of the anatomical region of interest, illustrating the 2D and 3D images augmented with annotations that provide visual indications of positions of the anatomical region of interest from which the 2D images are reconstructed. As illustrated in FIG. 5, each of the multiple images comprises one or more annotations providing a visual indication of a spatial relationship of one of the multiple images relative to another one of the multiple images. The one or more annotations may include a highlighted marking or the like.

In the illustrated embodiment, the 3D image comprises two annotations separately associated with each of the two 2D images, wherein each of the two annotation provides a visual indication of a position of the anatomical region of interest from within the 3D view that is associated with a 2D view of a respective 2D image. Similarly, each of the 2D images comprises an annotation associated with one another. For example, an annotation in a first one of the 2D images (e.g., 2D image providing a phased array visualization) provides a visual indication of a position of the anatomical region of interest from within the 2D view of the first one of the 2D images that is associated with the 2D view of a second one of the 2D images (e.g., 2D image providing a circumferential radial visualization). Similarly, an annotation in the second one of the 2D images provides a visual indication of a position of the anatomical region of interest from within the 2D view of the second one of the 2D images that is associated with the 2D view of the first one of the 2D images.

In some embodiments, the console 14 may be configured to augment the multiple images with the one or more annotations based, at least in part, on user input with the system. For example, the user may be able to select the specific portion of the region of interest from which the 2D images are to be reconstructed and further select whether the spatial relationship between the 2D and 3D images is to be highlighted.

For example, an interactive interface operably associated with the console is configured to allow a user to further select the type of annotation (i.e., select color, visibility, intensity, blurring, or the like) for highlighting the relationship between images. It should be noted that the annotations (i.e., highlighting or the like) may be permanently visible or may be associated with specific user input, such as changing of views, hovering of a mouse pointer over specific image regions, or other heuristics that indicate that highlighting may be useful at a given moment. By providing a user with the ability to freely select a visual cue of their choice, the system of the present invention enables a more intuitive relationship and interpretation by the user. The highlighting enables the user to make a more meaningful mental link between 3D and 2D views of an anatomical region of interest, thereby allowing a user to work with the data intuitively, while also providing a significant added value by providing the additional 3D view in real-, or near-real, time while data is updating.

It should further be noted that the 2D views can be reconstructed at arbitrary positions from the 3D views. Additionally, or alternatively, the 2D and 3D views can show the same information but reconstructed differently. In other words, both the 3D and 2D views may be reconstructed from the same raw data, or show different data derived from the raw data or data from other imaging modalities registered to it, or any combination of those various data sets).

In some embodiments, the console 14 is configured to receive the full circumferential, 3D image data in real-time or near real-time and the console is configured to reconstruct the multiple images in real-time or near real-time, based, at least in part, on user input and/or predefined protocols.

Accordingly, the system provides a user with an intuitive understanding of the interrelation of views and orientation within the acquired 3D data, thereby overcoming the limitations plaguing current imaging systems (i.e., 2D and 3D US imaging), while providing the advantages associated with each. In particular, the relationship between 2D and 3D views mimics current clinical imaging probes (e.g. linear/curvilinear/phased/rotational) in both appearance as well as interaction. In particular, users can digitally steer and/or navigate the 2D views (i.e., by way of interacting with the 3D volume via an interface) without requiring the manual maneuvering of the ultrasound probe, which can be cumbersome and prone to error.

As used in any embodiment herein, the term “module” may refer to software, firmware and/or circuitry configured to perform any of the aforementioned operations. Software may be embodied as a software package, code, instructions, instruction sets and/or data recorded on non-transitory computer readable storage medium. Firmware may be embodied as code, instructions or instruction sets and/or data that are hard-coded (e.g., nonvolatile) in memory devices. “Circuitry”, as used in any embodiment herein, may comprise, for example, singly or in any combination, hardwired circuitry, programmable circuitry such as computer processors comprising one or more individual instruction processing cores, state machine circuitry, and/or firmware that stores instructions executed by programmable circuitry. The modules may, collectively or individually, be embodied as circuitry that forms part of a larger system, for example, an integrated circuit (IC), system on-chip (SoC), desktop computers, laptop computers, tablet computers, servers, smartphones, etc.

Any of the operations described herein may be implemented in a system that includes one or more storage mediums having stored thereon, individually or in combination, instructions that when executed by one or more processors perform the methods. Here, the processor may include, for example, a server CPU and/or GPU, a mobile device CPU and/or GPU, and/or other programmable circuitry.

Also, it is intended that operations described herein may be distributed across a plurality of physical devices, such as processing structures at more than one different physical location. The storage medium may include any type of tangible medium, for example, any type of disk including hard disks, floppy disks, optical disks, compact disk read-only memories (CD-ROMs), compact disk rewritables (CD-RWs), and magneto-optical disks, semiconductor devices such as read-only memories (ROMs), random access memories (RAMs) such as dynamic and static RAMs, erasable programmable read-only memories (EPROMs), electrically erasable programmable read-only memories (EEPROMs), flash memories, Solid State Disks (SSDs), magnetic or optical cards, or any type of media suitable for storing electronic instructions. Other embodiments may be implemented as software modules executed by a programmable control device. The storage medium may be non-transitory.

As described herein, various embodiments may be implemented using hardware elements, software elements, or any combination thereof. Examples of hardware elements may include processors, microprocessors, circuits, circuit elements (e.g., transistors, resistors, capacitors, inductors, and so forth), integrated circuits, application specific integrated circuits (ASIC), programmable logic devices (PLD), digital signal processors (DSP), field programmable gate array (FPGA), logic gates, registers, semiconductor device, chips, microchips, chip sets, and so forth.

Reference throughout this specification to “one embodiment” or “an embodiment” means that a particular feature, structure, or characteristic described in connection with the embodiment is included in at least one embodiment. Thus, appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

The term “non-transitory” is to be understood to remove only propagating transitory signals per se from the claim scope and does not relinquish rights to all standard computer-readable media that are not only propagating transitory signals per se. Stated another way, the meaning of the term “non-transitory computer-readable medium” and “non-transitory computer-readable storage medium” should be construed to exclude only those types of transitory computer-readable media which were found in In Re Nuijten to fall outside the scope of patentable subject matter under 35 U.S.C. § 101.

The terms and expressions which have been employed herein are used as terms of description and not of limitation, and there is no intention, in the use of such terms and expressions, of excluding any equivalents of the features shown and described (or portions thereof), and it is recognized that various modifications are possible within the scope of the claims. Accordingly, the claims are intended to cover all such equivalents.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

1. An ultrasound imaging system comprising a console configured to be operably associated with an imaging device, wherein the console comprises a hardware processor coupled to non-transitory, computer-readable memory containing instructions executable by the processor to cause the console to:
receive three-dimensional (3D) image data from an ultrasound imaging device; and
dynamically reconstruct multiple images from the 3D image data, the multiple images comprising at least one 3D image providing a 3D view of an anatomical region of interest and one or more corresponding two-dimensional (2D) images providing 2D views of the anatomical region of interest and spatially related to the 3D view.

2. The system of claim 1, wherein each of the multiple images comprises at least one of a slice-based image and a volume-based image.

3. The system of claim 1, wherein the at least one 3D image provides a full circumferential, 360-degree visualization of the anatomical region of interest.

4. The system of claim 3, wherein the at least one 3D image provides an orbital volume visualization of the anatomical region of interest.

5. The system of claim 1, wherein the one or more 2D images provides at least one of a phased array visualization and circumferential radial visualization of the anatomical region of interest.

6. The system of claim 5, wherein the one or more 2D images provide multiple phased array views of the anatomical region of interest.

7. The system of claim 1, wherein each of the multiple images comprises one or more annotations providing a visual indication of a spatial relationship of one of the multiple images relative to another one of the multiple images.

8. The system of claim 7, wherein the multiple images comprise a 3D image and two or more 2D images spatially related to the 3D image.

9. The system of claim 8, wherein the 3D image comprises annotations separately associated with each of the two or more 2D images, wherein each annotation provides a visual indication of a position of the anatomical region of interest from within the 3D view that is associated with a 2D view of a respective 2D image.

10. The system of claim 8, wherein each of the 2D images comprises an annotation associated with one another.

11. The system of claim 10, wherein an annotation in a first one of the 2D images provides a visual indication of a position of the anatomical region of interest from within the 2D view of the first one of the 2D images that is associated with the 2D view of a second one of the 2D images.

12. The system of claim 11, wherein an annotation in a second one of the 2D images provides a visual indication of a position of the anatomical region of interest from within the 2D view of the second one of the 2D images that is associated with the 2D view of the first one of the 2D images.

13. The system of claim 7, wherein the one or more annotations comprises a highlighted marking, wherein the highlighted marking comprises a shade or color having a contrasting appearance relative to surrounding portion of a respective image upon which the annotation is applied.

14. The system of claim 7, wherein the console is configured to augment the multiple images with the one or more annotations based, at least in part, on user input with the system.

15. The system of claim 1, wherein the console is configured to receive the full circumferential, 3D image data in real-time or near real-time and the console is configured to reconstruct the multiple images in real-time or near real-time, based, at least in part, on user input and/or predefined protocols.

16. The system of claim 1, wherein the ultrasound imaging device comprises a catheter-based ultrasound imaging device comprising a catheter including a rotatable ultrasound transducer array provided thereon configured to transmit ultrasound pulses to, and receive echoes of the ultrasound pulses from, intravascular and/or intracardiac tissue, wherein the image data is in the form of reflected signal data based on received echoes of the ultrasound pulses from the intravascular and/or intracardiac tissue.

17. The system of claim 16, wherein the console is further configured to:
process the reflected signal data using at least one of a functional imaging algorithm and an anatomical imaging algorithm to extract associated functional and anatomical parameter data of the anatomical region of interest and reconstruct the multiple images from the extracted functional and/or anatomical parameter data; and
output, via a display, the reconstructed multiple images to an operator depicting visualization of the anatomical region of interest.

18. The system of claim 17, wherein the functional parameter data comprises at least one of tissue perfusion, tissue motion, tissue stiffness or elasticity, tissue strain, tissue anisotropy, tissue coherence, specific statistic tissue parameters modeled by statistical distributions, textural parameters of the tissue, and spectral and frequency-based parameters of the tissue, and blood flow, wherein the functional parameter data is indicative of a characterization of tissue at the anatomical region of interest and the anatomical parameter data comprises at least one of spatial and geometrical relationship of tissue at the anatomical region of interest, and the tissue characterization comprises at least one of tissue type, tissue health, tissue depth, lesion formation in the tissue as a result of an ablation procedure, and lesion depth in the tissue.

19. The system of claim 17, wherein the console is configured to output, via the display, the reconstructed multiple images in response to user input with a user interface operably associated with the console.

20. The system of claim 17, wherein the console is configured to output, via the display, the reconstructed multiple images based, at least in part, on running an algorithm determining one or more suggested views to displayed to the user based on the anatomical region of interest.
